(19) United States
(12) Patent Application Publication
(10) Pub. No.: US 2026/0243728 A1
(43) Pub. Date: Aug. 20, 2026

(54) SYSTEM AND METHOD RELATING TO AN ELECTRIC FIELD AT A LOCATION IN A VOLUME OF AN ELECTROLYTIC MEDIUM

(71) Applicant: IMPERIAL COLLEGE INNOVATIONS LIMITED, LONDON (GB)

(72) Inventors: JEAN RINTOUL, LONDON (GB); NIR GROSSMAN, LONDON (GB)

(73) Assignee: IMPERIAL COLLEGE INNOVATIONS LIMITED, LONDON, EN (GB)

(21) Appl. No.: 19/159,345

(22) PCT Filed: Feb. 23, 2024
(86) PCT No.: PCT/GB2024/050507

§ 371 Date: Aug. 25, 2025

(30) Foreign Application Priority Data
Feb. 24, 2023 (GB) 2302755.0

(51) Int. Cl. G01N 27/60

(52) U.S. Cl. CPC G01N 27/60

(57) ABSTRACT

The present disclosure relates to a method and system for measuring electric field at a location in a volume of an electrolytic medium and a method and system for applying an electric field to a location in a volume of an electrolytic medium. An aspect of the disclosure provides a method of determining an electric field at a location in a volume of an electrolytic medium, the method comprising: applying, to the location, acoustic energy having a first frequency; sensing an electric field for the volume to obtain a sensed electric field signal; determining, based on the sensed electric field signal and based on a reference signal having the first frequency, the electric field at the location, the electric field at the location having a second frequency.

### FIELD OF DISCLOSURE

The present disclosure relates to a method and system for measuring electric field at a location in a volume of an electrolytic medium and a method and system for applying an electric field to a location in a volume of an electrolytic medium.

### SUMMARY

The present invention is defined by the appended independent claims. Optional features are set out in the appended dependent claims.

An aspect of the disclosure provides a method of determining an electric field at a location in a volume of an electrolytic medium, the method comprising: applying, to the location, acoustic energy having a first frequency; sensing an electric field for the volume to obtain a sensed electric field signal; determining, based on the sensed electric field signal and based on a reference signal having the first frequency, the electric field at the location, the electric field at the location having a second frequency.

Embodiments advantageously provide a non-intrusive method of determining electric field (or electric current) at a location in a volume of an electrolytic medium. For example, damage to the electrolytic medium to gain access the location to measure the electric field at the location may be avoided.

The sensed electric field signal may have a plurality of frequency components, the frequency components comprising a third frequency wherein the third frequency is the difference of the first frequency and the second frequency.

Determining the electric field at the location may comprise demodulating the sensed electric field signal using the reference signal. For example, demodulating the sensed electric field signal comprises mixing the sensed electric field signal with the reference signal to obtain a mixed signal and low pass filtering the mixed signal. Advantageously, a third frequency (e.g. the difference between the first frequency and the second frequency) may be isolated. The demodulation may comprise I-Q demodulation.

The acoustic energy may be focused on the location. The acoustic energy may comprise ultrasound. The acoustic energy may be applied by a phased array transducer. Advantageously, acoustic energy can be applied to a specific location in the electrolytic volume and, in turn, a local electric field can be measured at the specific location.

The method may comprise controlling the acoustic energy to scan the position of the location about the volume while sensing the electric field for the volume thereby to determine the electric field at a plurality of locations in the volume.

An aspect of the disclosure provides an system for determining electric field at a location in a volume of an electrolytic medium, the system comprising: a source of acoustic energy configured to deliver acoustic energy to the location at a first frequency; an electric field sensor configured to sense electric field for the volume to provide a sensed electric field signal; and, a signal processor configured to determine the electric field at the location based on the sensed electric field and based on a reference signal having the first frequency.

Embodiments advantageously provide a non-intrusive system for determining electric field (or electric current) at a location in a volume of an electrolytic medium. For example, damage to the electrolytic medium to gain access the location to measure the electric field at the location may be avoided.

The signal processor may comprise a demodulator configured to mix the sensed electric field signal with the reference signal to provide a mixed signal. Optionally the demodulator comprises an I-Q demodulator. The signal processor may comprise a low pass filter, arranged to filter the mixed signal and having a cut-off frequency selected to exclude frequencies greater than or equal to the first frequency. Advantageously, a third frequency (e.g. the difference between the first frequency and the second frequency) may be isolated.

The source of acoustic energy may comprise an ultrasound source. The ultrasound source may be configured to focus the ultrasound energy on the location. The source of acoustic energy may comprise a phased array acoustic transducer.

An aspect of the disclosure provides a method of providing electric field to a location in a volume of electrolytic medium the method comprising: applying acoustic energy to the location at a first frequency; and applying electric field at a second frequency to the volume thereby to provide, at the location, electric field having a plurality of frequency components, the plurality of frequency components comprising at least one of: a third frequency component having a frequency of the difference between the first frequency and the second frequency; and a fourth frequency component having a frequency of the sum of the first frequency and the second frequency.

Embodiments advantageously provide a non-intrusive method of applying electric field (or electric current) at a location in a volume of an electrolytic medium. For example, damage to the electrolytic medium to gain access the location to apply electric field at the location may be avoided.

The acoustic energy may be focused on the location. The acoustic energy may comprise ultrasound. The acoustic energy may be applied by a phased array transducer. Advantageously, acoustic energy can be applied to a specific location in the electrolytic volume and, in turn, a local electric field can be measured at the specific location.

The method may comprise controlling the acoustic energy to scan the position of the location about the volume while applying the electric field at the second frequency thereby to provide said electric field at a plurality of locations in the volume.

The second frequency may be selected to provide a weaker interaction with the electrolytic medium than at least one of the third frequency component and the fourth frequency component.

An aspect of the disclosure provides a system for providing electric field to a location in a volume of electrolytic medium the system comprising: a source of acoustic energy configured to apply, to the location, acoustic energy at a first frequency, f₁; and an electric field applier configured to apply electric field to the volume at a second frequency, f₂, thereby to provide, at the location, electric field having a plurality of frequency components, the plurality of frequency components comprising at least one of: a) a third frequency component, f₃, having a frequency of the difference between the first frequency and the second frequency; and b) a fourth frequency component, f₄, having a frequency of the sum of the first frequency and the second frequency.

In an embodiment, this aspect of the disclosure may be configured to convert the frequency of an electrically applied electric field, applied to a volume, to the difference frequencies, f₂ only at a specific location L within that volume, those difference frequencies corresponding to between the acoustic energy and fields respectively. It may also be employed configured to convert the frequency of an electrically applied electric field, applied to a volume, to the sum frequencies, f₄ at the location L.

The frequency, f₂, at which the applied electric field is originally (electrically) applied to the volume may be selected to be outside a range of frequencies in which an interaction with the medium takes place. The apparatus may be configured so that the converted frequency (e.g., the difference frequency fs or the sum frequency f₄) however is inside the range of frequencies in which an interaction with the medium takes place. The apparatus and methods of the present disclosure can use this effect remotely and focally generate an interaction between the medium the electric field. For example, to provide an electric field which is active (interacts with the medium) only at the location in which the relevant acoustic energy is present.

In the case of excitable biological tissue such as (neurons and cardiac cells), the cells respond to electric fields up to a particular frequency range. Thus, by applying an electric field with a frequency higher than this responsive range to the volume of tissue as a whole, we can then use ultrasound to remotely convert the frequency to that in a responsive range only at the target region, achieving 3D focal stimulation.

Embodiments advantageously provide a non-intrusive system for applying electric field (or electric current) at a location in a volume of an electrolytic medium. For example, damage to the electrolytic medium to gain access the location to apply electric field at the location may be avoided.

The source of acoustic energy may be configured to focus the acoustic energy on the location. The acoustic energy may comprise ultrasound. The source of acoustic energy may comprise a phased array transducer. Advantageously, acoustic energy can be applied to a specific location in the electrolytic volume and, in turn, a local electric field can be measured at the specific location.

### BRIEF DESCRIPTION OF THE DRAWINGS

Some examples will now be described, by way of example only, which reference to figures, in which:

FIG. 1A illustrates an system and electrolytic medium, wherein the system is for determining electric field at a location L in a volume of the electrolytic medium;

FIG. 1B illustrates a schematic of an I-Q demodulator;

FIGS. 2A and 2B illustrate an indication of an actual electric field at the location L in an electrolytic medium;

FIGS. 2C and 2D illustrate an indication of a sensed electric field of a volume of an electrolytic medium 1;

FIGS. 2E and 2F illustrate an indication of the electric field at the location L;

FIG. 3 illustrates a flowchart which depicts a method for determining an electric field at a location in a volume of an electrolytic medium;

FIG. 4 illustrates an system and electrolytic medium, wherein the system is for providing electric field to a location L in a volume of the electrolytic medium;

FIG. 5A illustrates a time-domain representation of applied acoustic energy having the first frequency f₁;

FIG. 5B illustrates a time-domain representation of an applied electric field having the second frequency f₂;

FIG. 5C illustrates a time-domain representation of the local electric field at the location L of the electrolytic medium having a plurality of frequency components;

FIG. 6 illustrates a flowchart which depicts a method for applying an electric field at a location in a volume of an electrolytic medium.

In the drawings like reference numerals are used to indicate like elements.

### SPECIFIC DESCRIPTION

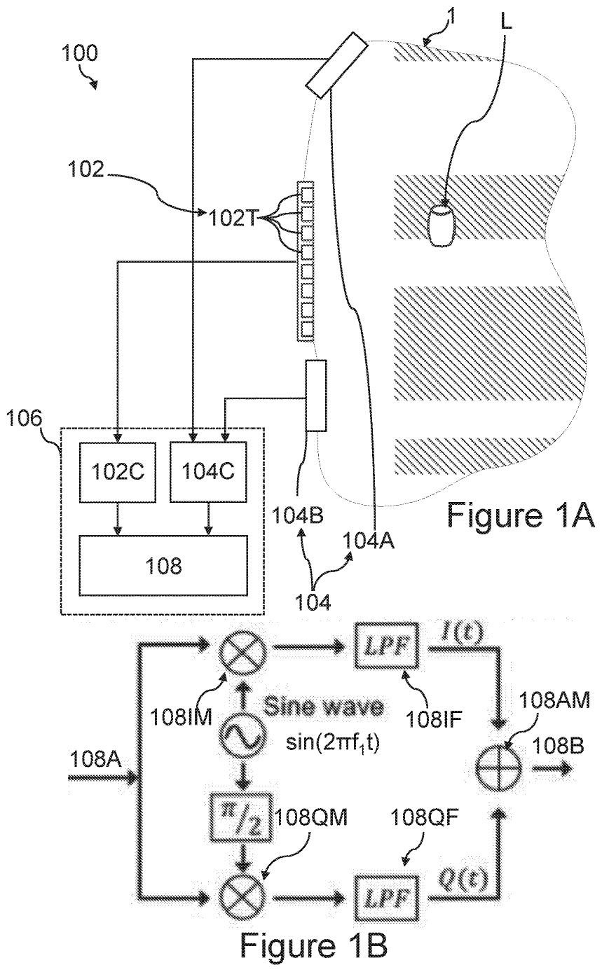

FIG. 1A illustrates an system 100 and electrolytic medium 1, wherein the system 100 is for determining electric field at a location L in a volume of the electrolytic medium 1. The system 100 comprises: a source of acoustic energy 102; an electric field sensor 104; and, a controller 106. The controller 106 comprises a signal processor component 108 and also an acoustic control component 102C and an electric field sensor component 104C.

An electric field (and corresponding current) may be present within the volume of the electrolytic medium 1. Depending on the nature of the medium this electric field may arise from a variety of source for example, where the medium comprises a living biological tissue it may arise from potentials which exist in nerve cells. Such electric field may be time varying. In the discussion which follows the frequency content of this electric field at the location L which in the volume is denoted f_{2.}

The electric field at location L, having this frequency content f₂, can be determined using the system 100. The system 100 may be coupled to the electrolytic medium 1 so that: the source of acoustic energy 102 can be used to deliver acoustic energy to the location L wherein the acoustic energy has a first frequency f₁; and, the electric field sensor 104 can be used to sense electric field for the volume of the electrolytic medium.

Without wishing to be bound by theory it is believed that, when the acoustic energy is applied to the electrolytic medium this affects the spatial distribution of charges in the electrolytic medium 1. A concomitant current and/or electric field effect may be ascribed to this acoustic induced behaviour of the charges in the medium. This acoustic induced electric field interacts with electric field already present at the location L. This interaction may give rise to an effect analogous to heterodyning. This heterodyning effect results in the electric field sensed by the electric field sensor 104 to comprise two frequency components, namely, a third frequency f₃ which is the difference between the first frequency f₁ and the second frequency f₂, and the fourth frequency f₄ which is the sum of the first frequency f₁ and the second frequency f₂. It will be appreciated in the context of the present disclosure that the electric field already existing at the location may not be a narrowband signal and so the frequency content denoted f₂ (second frequency) may actually comprise a number of different frequency components with corresponding effect on the resulting sum and difference frequencies.

By exploiting this effect and unmixing the signal of acoustic origin from the measured electric field for the volume the electric field at the location L can be determined as can its frequency content f_{2.}

To this end, the controller 106 comprises a signal processor 108 which receives the sensed electric field signal from the electric field sensor 104. The sensed electric field signal is demodulated (for example by I-Q demodulation) and the demodulated signal is filtered to determine the electric field at the location L and optionally its frequency content f₂. A more detailed description of the arrangement of system 100 as well as a detailed description of the system 100 in operation is set out below.

The source of acoustic energy 102 is connected to the controller 106. In particular, the source of acoustic energy 102 is connected to the acoustic control component 102C which forms part of the controller 106. As illustrated in FIG. 1A the source of acoustic energy 102 can be disposed in contact with the electrolytic medium 1 to thereby deliver acoustic energy to the location L at a first frequency f₁. The source of acoustic energy 102 may comprise a phased array acoustic transducer described in more detail herein.

In use, the source of acoustic energy 102 is attached to the electrolytic medium 1 to provide an acoustic coupling. The acoustic coupling may be improved by disposing a water-based gel between the source of acoustic energy 102 and the electrolytic medium 1. Disposing gel between the source of acoustic energy 102 and the electrolytic medium 1 may improve transmission of acoustic energy from the source 102 into the electrolytic medium 1.

The source of acoustic energy 102 sends a reference signal to the acoustic control component 102C and, in turn, the acoustic control component 102C sends the reference signal to the signal processor component 108. The reference signal is indicative the characteristics of the acoustic energy delivered by the source of acoustic energy 102 to the location L e.g. the reference signal is indicative of the first frequency f₁ (i.e. the frequency of the applied acoustic energy). Also the reference signal may be indicative of another property, such as the amplitude of the applied acoustic energy. The source of acoustic energy 102 may be an ultrasound source configured to emit acoustic energy having a frequency (i.e. a first frequency f₁) of, or around, 500 KHz.

Alternatively, the acoustic control component 102C may generate a reference signal based on a control signal sent to the source of acoustic energy e.g. the acoustic control component 102C may control the source of acoustic energy 102 to provide acoustic energy having a given frequency to the location L and based on this control, the acoustic control component 102C may generate a reference signal having the first frequency f_{1.}

The source of energy 102 illustrated in FIG. 1A is a phased array acoustic transducer. The phased array acoustic transducer 102 comprises a plurality of acoustic transducers 102T. The plurality of acoustic transducers 102T are disposed in an array, for example, a two-dimensional (2-D) array typically with an equal spacing between adjacent transducers in a given direction. The phased array acoustic transducer 102 is shown in cross-section in FIG. 1A but it will be appreciated that the arrangement of transducers 102T is repeated in a direction oblique to the page i.e. there are more transducers 102T disposed in the direction perpendicular to the page to form a 2D grid of transducers.

The acoustic energy emitted by the phased array acoustic transducer 102 can be directed and focused at a given location (e.g. the location L) by controlling the emission of acoustic energy from each of the acoustic transducers 102T. For example, the phased array acoustic transducer 102 can be operated to selectively superpose acoustic energy emitted from each of the acoustic transducers 102T in the array to thereby provide resultant acoustic energy.

The direction and position (i.e. the focal point) of the resultant acoustic energy is affected by emitting acoustic energy from each transducer 102T with any of a: time offset between emission of acoustic energy from a given acoustic transducer relative to neighbouring acoustic transducers; and, a phase offset between emission of acoustic energy from a given acoustic transducer relative to neighbouring acoustic transducers. Thus by selecting a suitable time and/or phase offset of each transducer 102T, acoustic energy (i.e. resultant acoustic energy) can be provided to a given location (e.g. location L).

The acoustic control component 102C is provided for controlling the source of acoustic energy 102. The acoustic control component 102C controls the amplitude and frequency of the acoustic energy emitted by the source 102. The acoustic control component 102C controls the direction and focal point source of acoustic energy emitted by the source 102, for example, offsetting the time of emission or phase of acoustic energy emitted from each acoustic transducer 102T in the phased array acoustic transducer.

The acoustic control component 102C in FIG. 1A is shown as part of controller 106 but it will be appreciated that in examples that the acoustic control component 102C may form part of the source of acoustic energy 102 e.g. the acoustic control component 102C may be separate from the controller 106.

The electric field sensor 104 is connected to the controller 106. In particular, the electric field sensor 104 is connected to the electric field sensor component 104C which forms part of the controller 106. The electric field sensor 104 comprises a first electric field sensor electrode 104A and a second electric field sensor electrode 104B (e.g. a measurement electrode and a reference electrode). As illustrated in FIG. 1A the electric field sensor 104 can be disposed in contact with the electrolytic medium 1 to thereby sense electric field for the volume i.e. the first electric field sensor electrode 104A and a second electric field sensor electrode 104B can be connected to the electrolytic medium 1.

The electric field sensor 104 is configured to generate a sensed electric field signal. The electric field sensor component 104C is provided for controlling the electric field sensor 104. Each of the first electric field sensor electrode 104A and the second electrode field sensor electrode 104B provide an indication of the sensed electric field to the electric field sensor component 104C, for example, an electric potential measured by each of the electrodes. The electric field sensor component 104C is configured to generate a sensed electric field signal indicative of the electric field of the volume of the electrolytic medium 1 based on the indication provided by the electric field sensor 104. The electric field sensor component 104C is configured to provide a sensed electric field signal to the signal processor component 108.

The electric field sensor component 104C in FIG. 1A is shown as part of controller 106 but it will be appreciated that in examples that the electric field sensor component 104C may form part of the electric field sensor 104 e.g. the electric field sensor component 104C. may be separate from the controller 106.

The sensed electric field signal may have a plurality of frequency components. One of the frequency components may have a third frequency f₃ which is the difference between the first frequency f₁ and the second frequency f₂. One of the frequency components may have a fourth frequency f₄ which is the sum of the first frequency f₁ and the second frequency f₂. As described herein, it will be appreciated in the context of the present disclosure that the electric field already existing at the location L may not be a narrowband signal and so the frequency content denoted f₂ (second frequency) may actually comprise a number of different frequency components with corresponding effect on the resulting sum and difference frequencies. In other words, if the frequency content denoted f₂ comprises a number of different frequency components, then the frequency content denoted f₃ and f₄ may each comprise a number of different frequencies.

The signal processor component 108 is connected to each of the source of acoustic energy 102 and the electric field sensor 104. In the present example, the signal processor component 108 is connected to these elements via the acoustic energy control component 102C and the electric field sensor control component 104C respectively. The signal processor component 108 is thereby configured to receive the reference signal indicative of the applied acoustic energy (e.g. having the first frequency f₁) and the sensed electric field signal indicative of the electric field of the volume of the electrolytic medium 1. The signal processor component 108 uses a process of demodulation to unmix the sensed electric field signal using the reference signal to obtain an indication of the electric field at the location L.

The signal processor component 108 is configured to determine the electric field at the location L based on the sensed electric field signal and based on the reference signal. The signal processor component 108 comprises a demodulator, in the present example, the demodulator is an I-Q demodulator 108D. The I-Q demodulator 108D is configured to obtain a signal indicative of the electric field at the location L by first extracting an in-phase (i.e. ‘I’) component and a quadrature (i.e. ‘Q’) component from the sensed electric field signal.

FIG. 1B illustrates a schematic of the I-Q demodulator 108D. The I-Q demodulator 108D comprises: a demodulator input 108A; an in-phase multiplicative mixer 108IM; a quadrature multiplicative mixer 108QM; an in-phase low pass filter 108IF; a quadrature low pass filter 108QF; an additive mixer 108AM; and a demodulator output 108B.

The demodulator input 108A is connected to the in-phase multiplicative mixer 108IM; a quadrature multiplicative mixer 108QM. The in-phase multiplicative mixer 108IM is connected to the in-phase low pass filter 108IF. The in-phase low pass filter 108IF is connected to the additive mixer 108AM. The quadrature multiplicative mixer 108QM is connected to the quadrature low pass filter 108QF. The quadrature low pass filter 108QF is connected to the additive mixer 108AM. The additive mixer 108AM is connected to the demodulator output 108B.

The sensed electric field signal is provided to the demodulator input 108A by the electric field sensor component 104C. The demodulator input 108A sends a copy of the sensed electric field signal to each of the in-phase multiplicative mixer 108IM and to the quadrature multiplicative mixer 108QM.

The reference signal is provided to the in-phase multiplicative mixer 108IM. The reference signal may be a sine wave having the frequency f₁ i.e. sin(2πf₁t). The in-phase multiplicative mixer 108IM multiplies the sensed electric field signal with the reference signal to provide an in-phase mixed signal. The in-phase mixed signal is provided to the in-phase low pass filter 108IF. The in-phase mixed signal has a plurality of components, one component having the third frequency f₃ and another component having the fourth frequency f₄ which are described in more detail herein.

The in-phase low pass filter 108IF is configured to remove components from the in-phase mixed signal which have a frequency above the first frequency f₁. The in-phase low pass filter 108IF removes components from the in-phase mixed signal which have a frequency above the first frequency f₁ to provide a filtered in-phase mixed signal I(t) which is provided to the additive mixer 108AM. The quadrature mixed signal has a plurality of components, one component having the third frequency f₃ and another component having the fourth frequency f₄ which are described in more detail herein.

The reference signal is provided to the quadrature multiplicative mixer 108QM. The quadrature multiplicative mixer adds a phase of π/2 radians to the reference signal. In the present example the reference signal is a sine wave having frequency f₁, therefore, the reference signal with the additional phase of π/2 radians is represented by a cosine wave having the first frequency i.e. cos(2πf₁t). The quadrature multiplicative mixer 108QM multiplies the sensed electric field signal with the reference signal with the π/2 phase difference to provide a quadrature mixed signal. The quadrature mixed signal is provided to the quadrature low pass filter 108QF.

The quadrature low pass filter 108QF is configured to remove components from the quadrature mixed signal which have a frequency above the first frequency f₁. The quadrature low pass filter 108QF removes components from the quadrature mixed signal which have a frequency above the first frequency f₁ to provide a filtered quadrature mixed signal Q(t) which is provided to the additive mixer 108AM. The filtered quadrature mixed signal Q(t) comprises a component having the third frequency f₃ which is the difference between the first frequency f₁ and the frequency component f₂.

The additive mixer 108AM adds the filtered in-phase mixed signal I(t) to the filtered quadrature mixed signal Q(t) to provide an indication of the electric field at the location L. The indication of the electric field at the location L may comprise at least one of: an indication of the frequency content f₂ of the electric field at the location L; an indication of the amplitude of the electric field at the location L.

To determine an electric field at location L in the electrolytic medium 1, the system 100 may be operated in the following manner. The source of acoustic energy 102 is attached to the electrolytic medium 1 to apply acoustic energy to the location L of the electrolytic medium 1. A gel is disposed between source of acoustic energy 102 and the electrolytic medium to improve transmission of the acoustic energy into the electrolytic medium. The two electric field sensor electrodes 104A and 104B are connected to the electrolytic medium to sense (e.g. measure) an electric field of the volume of the electrolytic medium 1.

After the source of acoustic energy 102 and the electric field sensor 104 are connected to the electrolytic medium 1, then the source of acoustic energy 102 is operated to apply acoustic energy having a first frequency f₁ to the location L in the volume of the electrolytic medium 1. During application of the acoustic energy to location L of the electrolytic medium by the source of acoustic energy 102, the electric field sensor 104 is operated to sense an electric field for the volume of the electrolytic medium 1 i.e. to obtain a sensed electric field signal.

The sensed electric field signal is then demodulated by the signal processor component 108 to isolate the contribution to the sensed electric field signal due to the electric field at location L in the electrolytic volume 1.

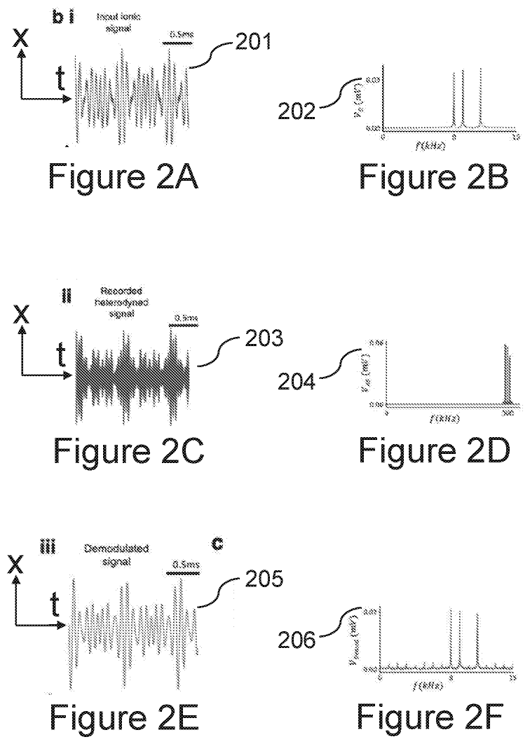

FIGS. 2A and 2B illustrate an indication of an actual electric field at the location L in the electrolytic medium; FIGS. 2C and 2D illustrate an indication of a sensed electric field of the electrolytic medium 1 (e.g. a sensed electric field signal); FIGS. 2E and 2F illustrate an indication of the electric field at the location L derived from the sensed electric field of the electrolytic medium (shown in FIGS. 2C and 2D).

FIGS. 2A, 2C, and 2E show a pair of axes, in particular, a time axis t and a displacement axis x. Thus indications 201, 203, and 205 in each of FIGS. 2A, 2C, and 2E variations of displacement of the magnitude of electric field with respect to time. FIGS. 2B, 2D, and 2F show a pair of axes, in particular, a frequency axis f and a displacement axis V. Thus indications 202, 204, and 206 in each of FIGS. 2B, 2D, and 2F show variations of displacement of the magnitude of electric field with respect to frequency.

FIG. 2A illustrates a time-domain representation 201 of the electric field at location L of the electrolytic medium 1. FIG. 2B illustrates a frequency-domain representation 202 of the electric field at location L of the electrolytic medium 1.

FIG. 2C illustrates a time-domain representation 203 of the electric field of the electrolytic medium 1. FIG. 2D illustrates a frequency-domain representation 204 of the electric field of the electrolytic medium 1. Either representation 203 or 204 may be considered a sensed electric field signal as described herein. As illustrated in FIGS. 2C and 2D, the indication of the electric field at the location L is modulated by a high frequency (e.g. see the shift in frequency in FIG. 2D). The source of the high frequency in the signal is the acoustic energy, the high frequency being the first frequency f₁. The acoustic energy is localised to the location L. It is the physical effect of the acoustic energy on the electrolytic medium 1 that causes the local electric field at location L (e.g. indicated in FIGS. 2A and 2B) to be modulated by the acoustic energy frequency f₁.

FIG. 2E illustrates a time-domain representation 205 of a measured the electric field at location L of the electrolytic medium 1. FIG. 2F illustrates a measured frequency-domain representation 206 of the electric field at location L of the electrolytic medium 1. By comparing FIG. 2A with 2E and FIG. 2B with 2F it will be appreciated that the measured electric field at the location L corresponds to the actual electric field at location L.

The measured electric filed at the location L is derived from the sensed electric field signal for the volume of the electrolytic medium 1 i.e. the representation 205 is obtained by demodulating representation 203 and likewise the representation 206 is obtained by demodulating representation 204.

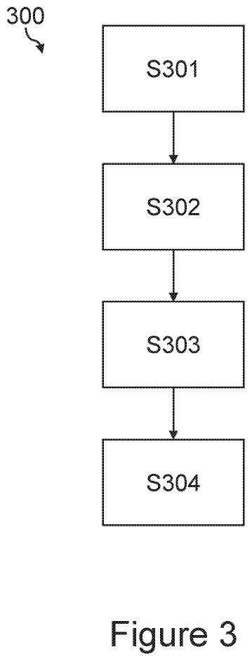

FIG. 3 illustrates a flowchart 300 which depicts a method for determining an electric field at a location in a volume of an electrolytic medium. The steps of the method are set out below.

The first step of the method is to apply, S301, to the location, acoustic energy having a first frequency f₁. The acoustic energy may be applied by a phased array transducer, which for example, may be used to focus the acoustic energy on the location. The applied acoustic energy may be ultrasound acoustic energy.

The second step of the method is to sense, S302, an electric field for the volume to obtain a sensed electric field signal. The sensed electric field signal may have a plurality of frequency components. One of the frequency components may comprise a third frequency f₃ wherein the third frequency f₃ is the difference between the first frequency f₁ and the second frequency f₂. One of the frequency components may comprise a fourth frequency f₄ wherein the fourth frequency f₄ is the sum of the first frequency f₁ and the second frequency f₂.

The third step of the method is to determine, S303, based on the sensed electric field signal and based on a reference signal having the first frequency, the electric field at the location, the electric field at the location having a second frequency.

Determining the electric field at the location may comprise demodulating the sensed electric field signal using the reference signal, for example, using I-Q demodulation.

The reference signal may be indicative of the amplitude and frequency of the applied acoustic energy. The sensed electric field signal may be indicative of the amplitude and frequency of the electric field at the location L.

The sensed electric field signal may be mixed with the reference signal to generate a mixed signal. The sensed electric field signal may be mixed with the reference signal using a multiplicative mixer to produce a mixed signal. The mixed signal may have a plurality of components. One of the components of the mixed signal has a frequency equal to the a third frequency f₃ wherein the third frequency f₃ is the difference between the first frequency f₁ and the second frequency f₂. One of the components of the mixed signal may comprise a fourth frequency f₄ wherein the fourth frequency f₄ is the sum of the first frequency f₁ and the second frequency f₂.

The mixed signal may be filtered to isolate the component of the mixed signal with the third frequency f₃, for example, the mixed signal may be filtered using a low pass filter (e.g. configured to remove frequencies higher than the first frequency f₁). Based on the isolated third frequency f₃ and the first frequency f₁, the second frequency f₂ can be determined e.g. f₂=f₃+f₁.

An optional step of the method comprises controlling, S304, the acoustic energy to scan the position of the location about the volume while sensing the electric field for the volume thereby to determine the electric field at a plurality of locations in the volume.

The above method is described as a series of discrete steps. However, the steps need not be discrete. The method may be performed continuously, for example, controlling the acoustic energy to scan the position of the location about the volume while sensing the electric field for the volume thereby to determine the electric field at a plurality of locations in the volume may be performed on a continuous basis e.g. to thereby map local electric field for the whole electrolytic medium. The method described above and depicted in FIG. 3 may be performed by the system illustrated in FIG. 1A.

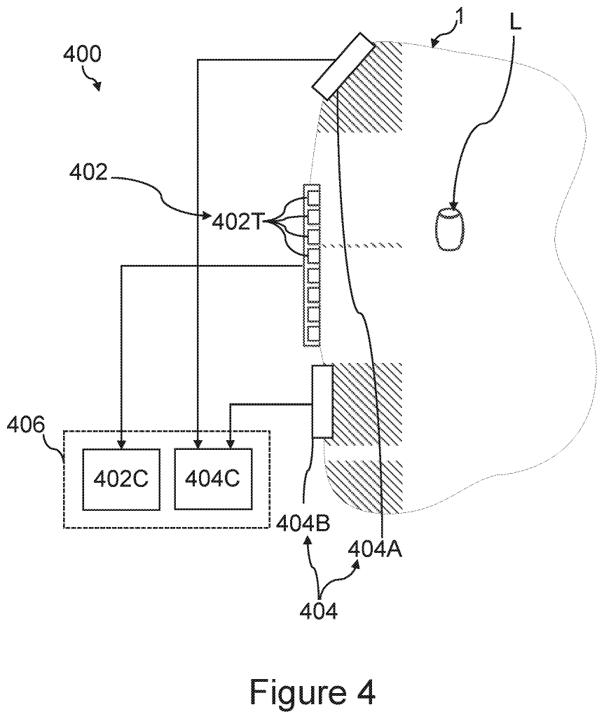

FIG. 4 illustrates a system 400 for providing electric field to a location L in a volume of an electrolytic medium 1.

The system 400 comprises: a source of acoustic energy 402; an electric field applier 404; and, a controller 406. The controller 406 comprises an acoustic control component 402C and an electric field control component 404C.

An electric field (and corresponding current) may be applied to a location L of a volume of the electrolytic medium 1 by exploiting an coupling between electric field applied to the volume and acoustic energy applied to the location L. The electric field applied to the volume as well as the electric field applied to the location L (i.e. through the electroacoustic coupling) may be time varying. In the discussion which follows the acoustic energy is applied at a first frequency denoted f₁ and the frequency content of the electric field applied to the volume is denoted f₂ (i.e. second frequency). Applying electric field with known properties (e.g. amplitude and frequency) to a specific location may be useful in a variety of circumstances, for example, where the medium comprises a biological tissue it may permit potentials which exist in tissues to be modified.

The electric field applied at location L can be provided using the system 400. The system 400 may be coupled to the electrolytic medium 1 so that: the source of acoustic energy 402 can be used to deliver acoustic energy to the location L wherein the acoustic energy has a first frequency f₁; and, the electric field applier 404 can be used to apply electric field to the volume of the electrolytic medium 4 with a frequency f₂.

Without wishing to be bound by theory it is believed that, when the acoustic energy is applied to the electrolytic medium this affects the spatial distribution of charges in the electrolytic medium 4. A concomitant current and/or electric field effect may be ascribed to this acoustic induced behaviour of the charges in the medium. This acoustically induced electric field is believed to interact with the electric field present at the location L due to the electric field applied to the volume by the electric field applier 404. This interaction may give rise to an effect analogous to heterodyning. This heterodyning effect results in an electric field at the location L to comprise two frequency components, namely:
1. frequency components having a third frequency content, f₃, which represent the difference frequencies—e. g, the differences between: (a) the frequency of the acoustically induced electric field (first frequency f₁) at the location, L; and (b) the frequency of the applied electric field (second frequency f₂) for the volume, and
2. frequency components having a fourth frequency content, f₄ which represent the sum frequencies—e.g., the sum of the frequency of the acoustically induced electric field (first frequency f₁) and the frequency of the applied electric field (second frequency f₂) for the volume.

It will be appreciated in the context of the present disclosure that the electric field applied to the volume may not be a narrowband signal and so the frequency content denoted f₂ (second frequency) may actually comprise a number of different frequency components with corresponding effect on the resulting sum and difference frequencies.

By exploiting this effect an electric field with known properties (e.g. amplitude and frequency) can be applied to a specific location L within the volume of the electrolytic medium 1. For example at least one of the third frequency component f₃ and the fourth frequency component f₄ is selected to have an affect on the electrolytic medium e.g. to stimulate nerve stimulation therein or to provide a current therein.

It can therefore be seen that it is possible to use acoustic energy to remotely and focally convert the frequency of an applied electric field (to the sum and difference frequencies of the acoustic energy and fields respectively).

If the frequency at which the electric field is originally applied to the volume is inert, in the sense of being outside the range of frequencies in which interaction with the medium takes place, but the converted frequency (e.g., the difference frequency) is not, the apparatus and methods of the present disclosure can use this effect remotely and focally generate an interaction between the medium the electric field. For example to provide an electric field which is active (interacts with the medium) only at the location in which the relevant acoustic energy is present.

In the case of excitable biological tissue such as (neurons and cardiac cells), the cells respond to electric fields up to a particular frequency range. Thus, by applying an electric field with a frequency higher than this responsive range to the volume of tissue as a whole, we can then use ultrasound to remotely convert the frequency to that in a responsive range only at the target region, achieving 3D focal stimulation.

To this end, the controller 406 comprises an acoustic control component 402C and an electric field control component 404C to permit control of the source of acoustic energy 402 and the electric field applier 404. A more detailed description of the arrangement of system 100 as well as a detailed description of the system 100 in operation is set out below.

The source of acoustic energy 402 is connected to the controller 406. In particular, the source of acoustic energy 402 is connected to the acoustic control component 402C which forms part of the controller 406. As illustrated in FIG. 4 the source of acoustic energy 402 can be disposed in contact with the electrolytic medium 1 to thereby deliver acoustic energy to the location L at a first frequency f₁. The source of acoustic energy 402 may comprise a phased array acoustic transducer described in more detail herein.

In use, the source of acoustic energy 402 is attached to the electrolytic medium 1 to provide an acoustic coupling. The acoustic coupling may be improved by disposing a water-based gel between the source of acoustic energy 402 and the electrolytic medium 1. Disposing gel between the source of acoustic energy 402 and the electrolytic medium 1 may improve transmission of acoustic energy from the source 402 into the electrolytic medium 1.

The controller 406 may receive an input (e.g. from a user) indicative of the location L that an electric field is to be provided to, and the properties of the electric field to be provided (e.g. frequency and amplitude). In response to this input the acoustic control component 402C and electric field applier component 404C may generate an acoustic control signal and an electric field control signal respectively to thereby provide the electric field to the location L.

The electric field applier electrodes 404A and 404B may be connected to a voltage source. The voltage source may comprise a controller configured to permit the voltage applied to the electric field applier electrodes to be varied to specified parameters (e.g. to allow an amplitude, frequency and/or phase of the voltage to be selected).

Alternatively the electric field applier electrodes 404A and 404B may be connected to a current source. The current source may comprise a controller configured to permit the current applied to the electric field applier electrodes to be varied to specified parameters (e.g. to allow an amplitude, frequency and/or phase of the current to be selected).

For example, the input may comprise a desired electric field waveform or a desired electric field frequency and magnitude to be provided at the location L. The controller 406 may be configured to determine a suitable acoustic energy waveform and electric field waveform (e.g. amplitude, frequency and phase (e.g. to provide the field at the correct location L)) which may interact to provide the desired electric field at location L. In examples, the controller may comprise a demodulator or simulated demodulator (e.g. an IQ demodulator) configured to demodulate the desired electric field waveform into an acoustic component (e.g. which may be provided by the source of acoustic energy 402) and an electric component (e.g. which may be provided by the electric field applier 404). The source of acoustic energy 402 illustrated in FIG. 4 is a phased array acoustic transducer. The phased array acoustic transducer 402 comprises a plurality of acoustic transducers 402T. The plurality of acoustic transducers 402T are disposed in an array, for example, a two-dimensional (2-D) array typically with an equal spacing between adjacent transducers in a given direction. The phased array acoustic transducer 402 is shown in cross-section in FIG. 4 but it will be appreciated that the arrangement of transducers 402T is repeated in a direction oblique to the page i.e. there are more transducers 402T disposed in the direction perpendicular to the page to form a 2D grid of transducers. The source of acoustic energy 402 may be an ultrasound source configured to emit acoustic energy having a frequency (i.e. a first frequency f₁) of, or around, 500 KHz.

The acoustic energy emitted by the phased array acoustic transducer 402 can be directed and focused at a given location (e.g. the location L) by controlling the emission of acoustic energy from each of the acoustic transducers 402T. For example, the phased array acoustic transducer 402 can be operated to selectively superpose acoustic energy emitted from each of the acoustic transducers 402T in the array to thereby provide resultant acoustic energy.

The direction and position (i.e. the focal point) of the resultant acoustic energy is affected by emitting acoustic energy from each transducer 402T with any of a: time offset between emission of acoustic energy from a given acoustic transducer relative to neighbouring acoustic transducers; and, a phase offset between emission of acoustic energy from a given acoustic transducer relative to neighbouring acoustic transducers. Thus by selecting a suitable time and/or phase offset of each transducer 402T, acoustic energy (i.e. resultant acoustic energy) can be provided to a given location (e.g. location L).

The acoustic control component 402C is provided for controlling the source of acoustic energy 402. The acoustic control component 402C controls the amplitude and frequency of the acoustic energy emitted by the source 402. The acoustic control component 402C controls the direction and focal point source of acoustic energy emitted by the source 402, for example, offsetting the time of emission or phase of acoustic energy emitted from each acoustic transducer 402T in the phased array acoustic transducer. The acoustic control component 402C is configured to generate and send an acoustic control signal to the source of acoustic energy 402. The acoustic control signal is indicative of the characteristics of the acoustic energy delivered by the source of acoustic energy 402 to the location L e.g. the signal is indicative of the first frequency f₁ (i.e. the frequency of the applied acoustic energy).

The acoustic control component 402C in FIG. 4 is shown as part of controller 406 but it will be appreciated that in examples that the acoustic control component 402C may form part of the source of acoustic energy 402 e.g. the acoustic control component 402C may be separate from the controller 406.

The electric field applier 404 is connected to the controller 406. In particular, the electric field applier 404 is connected to the electric field control component 404C which forms part of the controller 406. The electric field applier 404 comprises a first electric field applier electrode 404A and a second electric field applier electrode 404B. As illustrated in FIG. 4 the electric field sensor 404 can be disposed in contact with the electrolytic medium 1 to thereby apply an electric field to the volume i.e. the first electric field applier electrode 404A and a second electric field applier electrode 404B can be connected to the electrolytic medium 1. For example, a potential difference may be applied to each of the electrodes 404A and 404B to thereby provide an electric field therebetween.

The electric field control component 404C is provided for controlling the electric field applier 404. The electric field control component 404C controls the amplitude and frequency of the electric filed applied to the volume by the electric field applier 404. For example, the electric field control component 404C may control a potential difference to the electric field applier electrodes 404A and 404B. The electric field control component 404C is configured to generate and send an electric field control signal.

The electric field control component 404C in FIG. 4 is shown as part of controller 406 but it will be appreciated that in examples that the electric field control component 404C may form part of the electric field applier 404 e.g. the electric field applier controller 404C may be separate from the controller 406.

The electric field provided at the location L may have a plurality of frequency components. One of the frequency components may have a third frequency f₃ which is the difference between the first frequency f₁ and the second frequency f₂. One of the frequency components may have a fourth frequency f₄ which is the sum of the first frequency f₁ and the second frequency f₂. As described herein, it will be appreciated in the context of the present disclosure that the electric field applied to the volume may not be a narrowband signal and so the frequency content denoted f₂ (second frequency) may actually comprise a number of different frequency components with corresponding effect on the resulting sum and difference frequencies. In other words, if the frequency content denoted f₂ comprises a number of different frequency components, then the frequency content denoted f₃ and f₄ may each comprise a number of different frequencies.

To provide an electric field at location L in the electrolytic medium 1, the system 400 may be operated in the following manner. The source of acoustic energy 402 is attached to the electrolytic medium 1 to apply acoustic energy to the location L of the electrolytic medium 1. A gel is disposed between source of acoustic energy 402 and the electrolytic medium to improve transmission of the acoustic energy into the electrolytic medium. The two electric field applier electrodes 404A and 404B are connected to the electrolytic medium to apply an electric field of the volume of the electrolytic medium 1.

After the source of acoustic energy 402 and the electric field sensor 404 are connected to the electrolytic medium 1, then the source of acoustic energy 402 is operated to apply acoustic energy having a first frequency f₁ to the location L in the volume of the electrolytic medium 1. The source of acoustic energy 402 operates in response to the controller 406 (e.g. the acoustic energy controller 402C). During application of the acoustic energy to location L of the electrolytic medium by the source of acoustic energy 402, the electric field applier 404 is operated to apply an electric field to the volume of the electrolytic medium 1 wherein the applied electric field has a frequency content f₂.

The applied acoustic energy and electric fields interact to provide an electric field at location L. The electric field at location L has a plurality of frequency components, such as, the third frequency component f₃ and the fourth frequency component f₄. At least one of the third frequency component f₃ and the fourth frequency component f₄ is selected to affect the electrolytic medium in a desired way, for example, to stimulate a nerve cell at location L or to provide a current in the electrolytic medium 1.

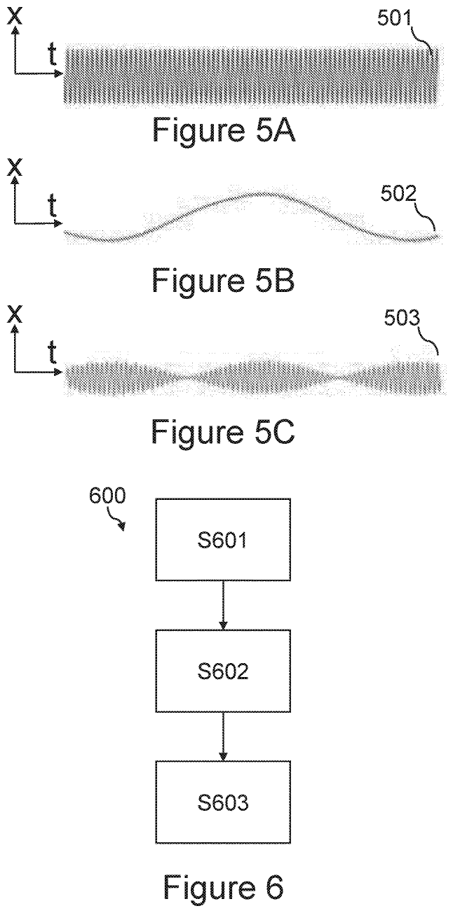

FIG. 5A illustrates a time-domain representation of applied acoustic energy 501 having the first frequency f₁. FIG. 5B illustrates a time-domain representation of an applied electric field 502 having the second frequency f₂. FIG. 5C illustrates a time-domain representation of the local electric field 503 at the location L of the electrolytic medium having a plurality of frequency components.

Each of FIG. 5A to 5C show a pair of axes, in particular, a time axis t and a displacement axis x. Thus signals 501 502 and 502 in each of FIGS. 5A to 5C show variations of displacement of the magnitude of the with respect to time.

As can be seen by comparing FIGS. 5A and 5B with 5C, an effect similar to heterodyning occurs at the location L due to the interaction between the electric field and the applied acoustic energy.

FIG. 6 illustrates a flowchart 600 which depicts a method for applying an electric field at a location in a volume of an electrolytic medium. The steps of the method are set out below.

The first step of the method is to apply, S601, acoustic energy to the location at a first frequency. The acoustic energy may be applied by a phased array transducer, which for example, may be used to focus the acoustic energy on the location. The applied acoustic energy may be ultrasound acoustic energy.

The second step of the method is to apply, S602, electric field at a second frequency to the volume thereby to provide, at the location, electric field having a plurality of frequency components, the plurality of frequency components comprising at least one of:
a) a third frequency component having a frequency of the difference between the first frequency and the second frequency; and
b) a fourth frequency component having a frequency of the sum of the first frequency and the second frequency.

The second frequency may be selected to provide a weaker interaction with the electrolytic medium than at least one of the third frequency component and the fourth frequency component.

The third step of the method is to control, S603, the acoustic energy to scan the position of the location about the volume while applying the electric field at the second frequency thereby to provide said electric field at a plurality of locations in the volume.

The frequency mixing property of the acoustoelectric effect results in generated electric fields that oscillate at the sum and difference frequencies. Hence, the temporal spatial pattern of the combined fields depends on the frequencies of the applied fields.

The above method is described as a series of discrete steps. However, the steps need not be discrete. The method may be performed continuously. The method described above and depicted in FIG. 6 may be performed by the system illustrated in FIG. 4. The electrolytic medium may be a part of an animal or human body, for example, part of an organ of an animal or human body such as a brain. The electrolytic medium may be a battery. If the electrolytic medium is a part of an animal or human body, then the location L may be a specific neuron or axon and thusly either: the electric field (or current) or the neuron or axon may be measured; or, an electric field (or current) may be applied to the neuron or axon.

The electrolytic medium may be disposed in a casing, in which case, the source of acoustic energy and the electric field sensor may be disposed in contact with the casing rather than being disposed in direct contact with the electrolytic medium. In examples wherein the electrolytic medium is a brain then the casing may be the head of the animal or human, such as the skull and the scalp.

It will be appreciated that the electric field sensor may comprise a monopolar arrangement e.g. having a reference electrode connected to a common voltage reference such as ground to which the electrolytic medium is also connected but bipolar arrangements may also be used, in which a differential voltage is measured between two electrodes connected to the medium. Direct physical connection to the medium may not be necessary provided that sufficient electrical coupling is present to allow sensing of electric field for the volume of the electrolytic medium. For example, one or both of the electrodes may not be in contact with the electrolytic medium.

It will be appreciated that the electric field applier (e.g. comprising the two electrodes used to apply electric field) may be arranged in the same way.

The electric field for the volume may be understood by reference to the two electrodes of the electric field sensor, for example the net electric field measured by such electrodes. It will be appreciated in the context of the present disclosure that the electronic current in conductive medium equals the product of the medium conductivity and electric field. Thus, electric field at a particular frequency (such as the difference and sum frequencies described herein) will also create current at those frequencies according to the conductivity of the medium

The method for determining an electric field may be modified so that instead it is for determining an electric current at a location in a volume of an electrolytic medium. The method may comprise applying, to the location, acoustic energy having a first frequency; sensing an electric field for the volume to obtain a sensed electric field signal; determining, based on the sensed electric field signal and based on a reference signal having the first frequency, the electric field at the location, the electric field at the location having a second frequency; and, determining a current at the location based on the electric field.

The current at the location can be determined from the electric field at the location by a number of routes. The current at the location may be determined based on Ohm's law I=V/R wherein V is the voltage at the location and R is the resistance at the location. The voltage V at the location is related to the electric field at the location E and the width of the location d by E=V/d, therefore, I=Ed/R. The current density at the location may be determined using the vector form of Ohm's law J=σE wherein J is the current density, o is the conductivity and E is the electric field at the location.

The controller 106 may be configured to calculate the current at the location L. The controller 106 may receive as an input (e.g. either from a person inputting data or a suitable measurement device) indicative of any of the parameters needed to compute the current, for example, the electric field at the location L (the controller may determine the electric field at the location as set out herein), the resistance of the location, the width of the location, the voltage across the location L and/or the conductivity of the location L (e.g. to calculate the current density J at the location L).

Without being bound theory, regions of compression and rarefaction modify density of electrolyte in the electrolytic medium at or close to the location L. The change in density of the electrolyte affects the charge density at the regions of compression and rarefaction. The charge density of the electrolyte may be greater at the regions of compression and may be lesser at the regions of rarefaction. The charge of the electrolyte produces an electric field. Changing the charge density of the electrolytes in the electrolytic medium changes the properties of the electric field. By applying acoustic energy to the location L, the charge density of the electrolytes at and around the location L is modified, which in turn modifies the electric field generated by the electrolytes. The electric field generated by the electrolytes is superposed with the applied electric field to generate a resultant electric field at location L, referred to herein as the local electric field at location L. The resultant electric field at is controllable by controlling the acoustic energy applied to location L.

The present disclosure describes a methods and systems for generating and measuring an electric field at a location in electrolytic medium using acoustic energy applied to the electrolytic medium. Therefore, the methods and systems described herein exploit a coupling between acoustic energy applied to an electrolytic medium and an electric field within the electrolytic medium. Without wishing to be bound by theory, the coupling between acoustic energy applied to an electrolytic medium and an electric field within the electrolytic medium may be described mathematically as follows.

An external electric field Eₒ(x, y, z) may induce an ionic current with a density J(x, y, z) an electrolytic medium (e.g. a weak electrolytic liquid) wherein the electrolytic medium has a conductivity σ(x, y, z). This may be expressed mathematically using Ohm's law J=σEₒ. If a current source is external to the electrolytic medium, then the divergence of the current density local to the volume of electrolytic medium is zero i.e. ∇·J=0. Combining this with Ohm's Law yields equation 1: ∇·σEₒ=0.

Acoustic energy applied to the electrolytic medium may be represented as a pressure field P(x, y, z). The acoustic energy applied to the electrolytic medium may change the ion concentration resulting in a conductivity σₐₑ(x, y, z) due to the acoustic energy and a corresponding electric field Eₐₑ due to the acoustic energy. Equation 1 can be rewritten as equation 2: ∇·((σ+σₐₑ)(Eₒ+Eₑₐ))=0

Ignoring high order terms, equation 2 can be rearranged to equation 3: ∇·(σEₑₐ)=−∇(kP)·(σEₒ) where k=σₐₑ/(Pσ). If the electrolytic medium and the applied electric field are homogeneous then equation 3 becomes equation 4: ∇Eₑₐ=−k∇P·Eₒ where ∇P is the gradient of the pressure field P. Therefore, equation 4 shows a coupling exists between the electric field and applied acoustic energy.

1. A method of determining an electric field at a location in a volume of an electrolytic medium, the method comprising:
applying, to the location, acoustic energy having a first frequency;
sensing an electric field for the volume to obtain a sensed electric field signal;
determining, based on the sensed electric field signal and based on a reference signal having the first frequency, the electric field at the location, the electric field at the location having a second frequency.

2. The method of claim 1 wherein the sensed electric field signal has a plurality of frequency components, the frequency components comprising a third frequency wherein the third frequency is the difference of the first frequency and the second frequency.

3. The method of claim 1 or 2 wherein determining the electric field at the location comprises demodulating the sensed electric field signal using the reference signal.

4. The method of claim 3 wherein the demodulation comprises I-Q demodulation.

5. The method of claim 3 or 4 wherein demodulating the sensed electric field signal comprises mixing the sensed electric field signal with the reference signal to obtain a mixed signal and low pass filtering the mixed signal.

6. The method of any preceding claim wherein the acoustic energy is focused on the location.

7. The method of claim 6 wherein the acoustic energy comprises ultrasound.

8. The method of any proceeding claim wherein the acoustic energy is applied by a phased array transducer.

9. The method of any preceding claim comprising controlling the acoustic energy to scan the position of the location about the volume while sensing the electric field for the volume thereby to determine the electric field at a plurality of locations in the volume.

10. An system for determining electric field at a location in a volume of an electrolytic medium, the system comprising:
a source of acoustic energy configured to deliver acoustic energy to the location at a first frequency;
an electric field sensor configured to sense electric field for the volume to provide a sensed electric field signal; and,
a signal processor configured to determine the electric field at the location based on the sensed electric field signal and based on a reference signal having the first frequency.

11. The system of claim 10 where in the signal processor comprises a demodulator configured to mix the sensed electric field signal with the reference signal to provide a mixed signal.

12. The system of claim 11 wherein the demodulator comprises an I-Q demodulator.

13. The system of claim 11 or 12 wherein the signal processor comprises a low pass filter, arranged to filter the mixed signal and having a cut-off frequency selected to exclude frequencies greater than or equal to the first frequency.

14. The system of any preceding claim wherein the source of acoustic energy comprises an ultrasound source.

15. The system of claim 14 wherein the ultrasound source is configured to focus the ultrasound energy on the location.

16. The system of any of claims 13 to 15 wherein the source of acoustic energy comprises a phased array acoustic transducer.

17. A method of providing electric field to a location in a volume of electrolytic medium the method comprising:
applying acoustic energy to the location at a first frequency; and
applying electric field at a second frequency to the volume thereby to provide, at the location, electric field having a plurality of frequency components, the plurality of frequency components comprising at least one of:
a) a third frequency component having a frequency of the difference between the first frequency and the second frequency; and
b) a fourth frequency component having a frequency of the sum of the first frequency and the second frequency.

18. The method of claim 17 wherein the acoustic energy is focused on the location.

19. The method of claim 17 or 18 wherein the acoustic energy comprises ultrasound.

20. The method of claim 17, 18 or 19 wherein the acoustic energy is applied by a phased array transducer.

21. The method of any of claims 17 to 20 comprising controlling the acoustic energy to scan the position of the location about the volume while applying the electric field at the second frequency thereby to provide said electric field at a plurality of locations in the volume.

22. The method of any of claims 17 to 21 wherein the second frequency is selected to provide a weaker interaction with the electrolytic medium than at least one of the third frequency component and the fourth frequency component.

23. An system for providing electric field to a location in a volume of electrolytic medium the system comprising:
a source of acoustic energy configured to apply, to the location, acoustic energy at a first frequency; and
an electric field applier configured to apply electric field to the volume at a second frequency thereby to provide, at the location, electric field having a plurality of frequency components, the plurality of frequency components comprising at least one of:
a) a third frequency component having a frequency of the difference between the first frequency and the second frequency; and
b) a fourth frequency component having a frequency of the sum of the first frequency and the second frequency.

24. The system of claim 23 wherein the source of acoustic energy is configured to focus the acoustic energy on the location.

25. The system of claim 23 or 24 wherein the acoustic energy comprises ultrasound.

26. The system of claim 23, 24, or 25 wherein the source of acoustic energy comprises a phased array transducer.
